(19) United States
(12) Patent Application Publication
(10) Pub. No.: US 2024/0238841 A1
(43) Pub. Date: Jul. 18, 2024

(54) Output Stage for High-Voltage Linear Transmitters

(71) Applicant: Texas Instruments Incorporated, Dallas, TX (US)

(72) Inventors: Raja Sekhar, Bangalore (IN); Ravikumar Pattipaka, Bangalore (IN); Sandeep Oswal, Bangalore (IN)

(21) Appl. No.: 18/192,863

(22) Filed: Mar. 30, 2023

Related U.S. Application Data
(60) Provisional application No. 63/438,933, filed on Jan. 13, 2023.

(51) Int. Cl. B06B 1/02; B06B 1/06; H03F 3/183

(52) U.S. Cl. CPC B06B 1/0207; B06B 1/06; H03F 3/183; A61B 8/4488; H03F 2200/03

(57) ABSTRACT

An ultrasonic transceiver system including a transducer, a receiver coupled to the transducer, a transmitter having an output terminal coupled to the transducer, and a transmit/receive switch configured to isolate the receiver from the transmitter during transmission. The transmitter includes an amplifier and an output stage. The output stage includes a source follower transistor having a drain coupled to a supply terminal, a gate coupled to an output of the amplifier, and a source coupled to an output terminal, along with a power stage transistor having a source coupled to the supply terminal, a gate coupled to the drain of the source follower transistor, and a drain coupled to the output terminal. The output stage further includes a parallel source follower transistor having a drain coupled to the supply terminal, a gate coupled to the input terminal, and a source coupled to the output terminal.

### CROSS-REFERENCE TO RELATED APPLICATIONS

This application claims priority, under 35 U.S.C. § 119(e), to U.S. Provisional Application No. 63/438,933, filed Jan. 13, 2023, said application incorporated herein in its entirety by this reference.

### BACKGROUND

This relates to linear amplifiers for generating high frequency signals at high voltages and currents, for example as applied to ultrasonic sensing systems.

Ultrasonic sensing systems, such as used in the field of medical imaging, involve actuating a transducer to produce ultrasonic waves into the target medium, and sensing reflections or echoes of the produced ultrasonic waves from structures in the target medium. In some implementations, the same transducer used to impart the ultrasonic waves is also used to sense reflections of those waves. In those implementations, the electronic circuitry of the ultrasonic system includes both transmitter and receiver circuitry, with a “transmit/receive” switch used to isolate the receiver from the transmitter during transmission.

Transmitter circuitry in ultrasonic sensing stages may include an output stage FIG. 1A illustrates an example of a prior art output stage ultrasonic transmitter circuitry. Output stage 100 in FIG. 1A is in the form of a “flipped” source-follower of n-channel MOS (NMOS) transistor 102N and p-channel MOS (PMOS) transistor 102P with gates coupled in common to input VIN and sources coupled in common to output node VOUT. The drain of NMOS source follower transistor 102N is coupled to the gate of power PMOS transistor 104P, and the drain of PMOS source follower transistor is coupled to the gate of power NMOS transistor 104N. The drains of power transistors 104P, 104N are coupled in common to capacitive load 105 at output node VOUT. Current source 103P biases the drain of NMOS source follower transistor 102N from positive supply terminal V+, and current source 103N biases the drain of PMOS source follower transistor 102P from negative supply terminal V−. The source of power PMOS transistor 104P is coupled to positive supply terminal V+, and the source of power NMOS transistor 104N is coupled to negative supply terminal V−. In high-voltage applications, the rail voltages at supply terminals V+ and V− may be quite high, for example as high as ±100 VDC.

### SUMMARY

In an example, transmitter circuitry for an ultrasonic transceiver system includes an amplifier and an output stage. The output stage includes a first source follower transistor having a drain coupled to a first supply terminal, a gate coupled to an output of the amplifier, and a source coupled to an output terminal, along with a first power stage transistor having a source coupled to the first supply terminal, a gate coupled to the drain of the first source follower transistor, and a drain coupled to the output terminal. The output stage further includes a first parallel source follower transistor having a drain coupled to the first supply terminal, a gate coupled to the input terminal, and a source coupled to the output terminal.

In another example, an ultrasonic transceiver system includes a transducer, ultrasonic receiver circuitry coupled to the transducer, ultrasonic transmitter circuitry having an output terminal coupled to the transducer, and a transmit/receive switch configured to isolate the ultrasonic receiver circuitry from the ultrasonic transmitter circuitry while the ultrasonic transmitter circuitry is transmitting. The ultrasonic transmitter circuitry includes an amplifier and an output stage, where the output stage includes a first source follower transistor having a drain coupled to a first supply terminal, a gate coupled to an output of the amplifier, and a source coupled to an output terminal, along with a first power stage transistor having a source coupled to the first supply terminal, a gate coupled to the drain of the first source follower transistor, and a drain coupled to the output terminal. The output stage further includes a first parallel source follower transistor having a drain coupled to the first supply terminal, a gate coupled to the input terminal, and a source coupled to the output terminal.

In another example, a transmitter output stage includes a first source follower transistor having a drain coupled to a first supply terminal, a gate coupled to an output of the amplifier, and a source coupled to an output terminal, along with a first power stage transistor having a source coupled to the first supply terminal, a gate coupled to the drain of the first source follower transistor, and a drain coupled to the output terminal. The output stage further includes a first parallel source follower transistor having a drain coupled to the first supply terminal, a gate coupled to the input terminal, and a source coupled to the output terminal.

Technical advantages enabled by one or more of these examples include a reduction in the time that output stage source follower transistors are in quasi-saturation, particularly in transmitters operating at high voltages, high currents, and high frequencies, while maintaining good stability and phase margin for the transmitter. These one or more examples also enable efficiencies in their implementation in an integrated circuit.

Other technical advantages enabled by the disclosed aspects will be apparent to those of ordinary skill in the art having reference to the following specification together with its drawings.

### BRIEF DESCRIPTION OF THE DRAWINGS

FIG. 1A is an electrical diagram, in schematic form, of a prior art output stage.

FIG. 1B is an example of a pole-zero plot of the transfer characteristic of the output stage of FIG. 1A.

FIG. 2A is an electrical diagram, in block form, of an example ultrasonic transceiver system.

FIG. 2B is an electrical diagram, in block form, of an example ultrasonic transmitter in the system of FIG. 2A.

FIG. 3 is a plot of drain current, transconductance, and gate-to-source capacitance versus gate-to-source voltage for a high voltage metal-oxide-semiconductor (MOS) transistor.

FIG. 4A is an electrical diagram, in schematic form, of an example transmitter output stage.

FIG. 4B is an electrical diagram, in schematic form, of an example upper half transmitter output stage.

FIG. 4C is a comparison plot of turn-on time for a power transistor in the output stage of FIG. 4B as compared with an output stage as shown in FIG. 1A.

The same reference numbers or other reference designators are used in the drawings to illustrate the same or similar (in function and/or structure) features.

### DETAILED DESCRIPTION OF EXAMPLE EMBODIMENTS

Piezoelectric ultrasonic transducers tend to be inefficient, from the standpoint of the mechanical ultrasonic energy generated from the electrical energy applied by the transmitter output circuitry. This inefficiency can require the transmitter circuitry to output signals at high voltages (e.g., on the order of 180 V peak-to-peak) and high currents (e.g., on the order of 6 A peak-to-peak) in order to generate the ultrasonic energy necessary for imaging. In addition, to attain high resolution images, the ultrasonic frequencies of interest can be as high as 25 MHz.

The reflections of interest to be sensed by the ultrasonic receiver from structures in the target medium generally correspond to the second harmonic of the imparted ultrasonic waves. The amplitude of these sensed reflections may be quite low as compared with the transmitter output, for example at levels on the order of 500 mV peak-to-peak. The low voltage of the received signals places stringent limits on the linearity of the transmitter, to minimize distortion in the transmitted signal that can mask the true reflections.

Stability of the transmitter circuitry in generating the desired high frequency signals at high voltage and current levels is also of significant concern in these systems.

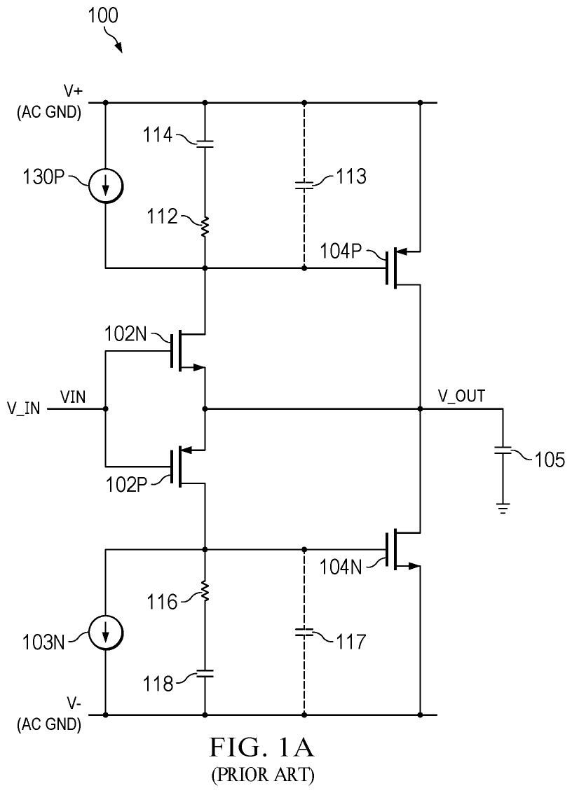

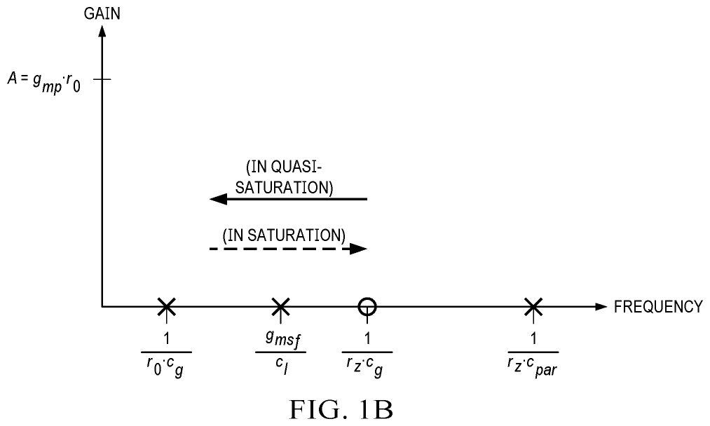

In the operation of output stage 100 of FIG. 1A, a high voltage signal (e.g., as high as 180V peak-to-peak) signal at the desired frequency (e.g., on the order of 5 MHz) is received at input VIN. Source follower transistors 102N, 102P turn on and off with the signal voltage to drive the voltage at output node VOUT accordingly. In rising half-cycles of the voltage at input VIN, the on state of NMOS source follower transistor 102N operates to discharge the gate of power PMOS transistor 104P, which turns on that transistor 104P to conduct current from supply terminal V+ to output node VOUT. Similarly, in falling half-cycles of the voltage at input VIN, the on state of PMOS source follower transistor 102P operates to charge the gate of power NMOS transistor 104N, which turns on that transistor 104N to conduct current from supply terminal V− to output node VOUT. Power transistors 104P and 104N are sized to produce the bulk of the output current applied to output node VOUT by output stage 100, while source follower transistors 102N and 102P need only be sized sufficiently to discharge and charge the gates of their corresponding power transistors 104P, 104N.

Output stage 100 as shown in FIG. 1A includes pole-zero compensation to provide the desired gain and phase margin at for the desired load conditions. This pole-zero compensation includes resistor 112 and capacitor 114 coupled in series between the gate of power PMOS transistor 104P and positive supply terminal V+, and resistor 116 and capacitor 118 coupled in series between the gate of power NMOS transistor 104N and negative supply terminal V−. For purposes of AC analysis of output stage 100, supply terminals V+, V− can be considered to be at “AC ground”. Parasitic gate to source capacitances of power transistors 104P, 104N are also present, as shown in shadow as capacitors 113, 117, respectively.

FIG. 1B illustrates the position of poles and zeroes along the frequency axis in the gain transfer function of output stage 100 as shown in FIG. 1A. Considering the transfer function of the top half (source follower NMOS transistor 102N and power PMOS transistor 104P) of output stage 100 when sourcing load current, the DC gain (A) of source follower output stage 100 can be considered as the product of the transconductance of power PMOS transistor 104P (gₘₚ) and a DC input impedance (r₀) at the gate of power PMOS transistor 104P. The dominant pole established at the gate of power PMOS transistor 104P is at a frequency defined by the reciprocal of the product of input impedance (r₀) and the capacitance (c_{g}) of compensation capacitor 114. A non-dominant “load” pole is established at a frequency defined by the transconductance of source follower NMOS transistor 102N (g_{msf}) divided by the capacitance (cₗ) of load 105. A higher frequency zero is at a frequency corresponding to the reciprocal of the product of capacitance c_{g} of compensation capacitor 114 and the resistance r_{z} of compensation resistor 112 (for the upper half; the lower half has a similar zero). A higher frequency pole is also present in this transfer function, but can typically be set at frequencies beyond those of interest in practical implementations. The lower half of output stage 100 (source follower PMOS transistor 102P and power NMOS transistor 104N) has a similar transfer function when sinking load current.

The transconductance of MOS transistors, including high-voltage MOS transistors such as DEMOS devices, tends to increase with increasing gate-to-source voltage. Accordingly, as higher gate-to-source voltages are applied to input VIN of output stage 100 to drive increasing load currents, the transconductance (g_{msf}) of the source follower transistors 102N, 102P tends to increase. These higher transconductances cause the non-dominant load pole in the pole-zero plot to move higher in frequency, which is of benefit in the linearity and stability at the intended load conditions.

Also, an effect referred to as quasi-saturation is known to occur in high voltage metal-oxide-semiconductor (MOS) transistors, such as drain-extended MOS (DEMOS) transistors, when operated at high drain current levels. This quasi-saturation effect is exhibited by a reduction in transconductance at high drain currents.

It is within this context that the embodiments described herein arise.

One or more examples are described in this specification as implemented ultrasonic transmitter circuitry, as it is contemplated that such implementation is particularly advantageous in that context. However, aspects of these examples may be beneficially applied in other applications in which a high voltage output signal is to be driven at high current and high frequencies. Accordingly, the following description is provided by way of example only, and is not intended to limit the true scope of this invention as claimed.

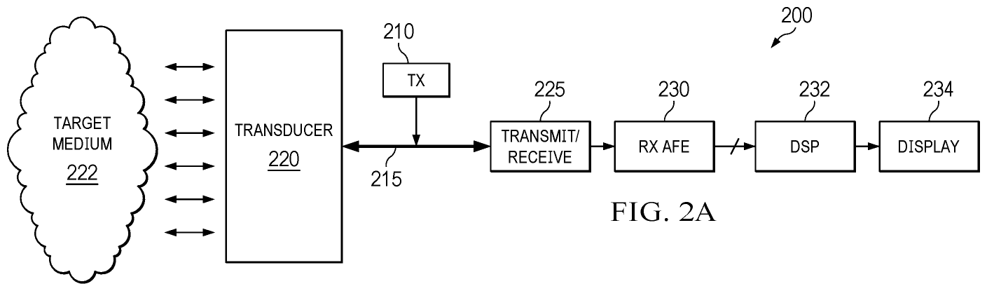

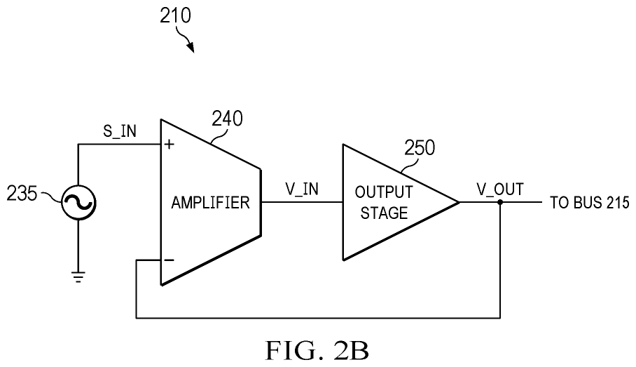

FIG. 2A illustrates an example of an ultrasonic transceiver system 200. Ultrasonic transceiver system 200 in this example, includes ultrasonic transmitter 210, signal bus 215, ultrasonic transducer 220, transmit/receive switch 225, receiver analog front end (AFE) 230, digital signal processor (DSP) 232, and display 234. Transmitter 210, transmit/receive switch 225, receiver AFE 230, and digital signal processor 232 may each be implemented as one or more integrated circuits. Alternatively, two or more of transmitter 210, transmit/receive switch 225, receiver AFE 230, and digital signal processor 232 may be implemented into the same integrated circuit.

In this example, ultrasonic transmitter 210 has an output coupled to ultrasonic transducer 220 by way of signal bus 215. Bus 215 may correspond to a cable and other conductors (e.g., printed circuit board traces) that electrically couple transducer 220 to transmitter 210 and transmit/receive switch 215. Ultrasonic transducer 220 may include one or more piezoelectric elements or capacitively-driven micro-machined ultrasonic transducers, which operate to convert electrical energy into mechanical energy (e.g., acoustic waves) and vice versa. In the context of medical imaging, for example, transducer 220 may be arranged as a two-dimensional array of piezoelectric elements to impart mechanical energy into a target medium 222 (e.g., a human body) in response to electrical signals output by transmitter 210 and communicated via bus 215.

As noted above, transducer 220 also operates to sense mechanical energy from the target medium and convert that mechanical energy into electrical signals. In this example, the mechanical energy sensed by transducer 220 may correspond to reflections of acoustic waves imparted by transducer 220 itself in response to electrical signals from transmitter 210. In system 200 of FIG. 2A, electrical signals generated by transducer 220 in response to sensing such reflections are output by transducer 220 onto bus 215.

Transmit/receive switch 225 is provided in system 200 of FIG. 2A between bus 215 and receiver AFE circuitry 230. Transmit/receive switch 225 is operable to isolate receiver AFE circuitry 230 from bus 215 during transmission periods and couple bus 215 to receiver AFE circuitry 230 during receive periods. In ultrasonic systems in which transducer efficiency is relatively low, the signals output by transmitter 210 may be at very high voltages and currents (e.g., 100V p-p at 6 A p-p) relative to the amplitude (e.g., 500 mV p-p) of the electrical signals output by transducer 220 in response to sensed reflections. The isolation provided by transmit/receive switch 225 can prevent the overdriving of input circuitry of receiver AFE 230 by the high voltage and current output of transmitter 210 in transmitting intervals. The particular placement of transmit/receive switch 225 may vary depending on the system architecture. For example, transmit/receive switch 225 may alternatively be placed between transmitter 210 and bus 215, or may alternatively be between transducer 220 and both of transmitter 210 and receiver AFE 230.

Receiver AFE 230 includes circuitry such as a low noise amplifier (LNA) for amplifying the electrical signals from transducer 220, receiver filters to minimize receive path noise, comparators and/or threshold detectors for measuring travel times of the acoustic reflections, analog-to-digital converter (ADC) circuitry, etc. as suitable for the particular implementation. In system 200 of FIG. 2A, receiver AFE 230 can output digital signals to digital signal processor (DSP) 232 for processing and display of results at display 234.

FIG. 2B illustrates an example architecture of transmitter 210. Transmitter 210 includes a signal source 235, an amplifier 240 (e.g., a linear amplifier), and an output stage 250. In this example, signal source 235 generates a time-varying signal S_IN, for example a sinusoidal or square wave signal at one or more frequencies. Signal S_IN is received at an input of linear amplifier 240. Linear amplifier 240 amplifies input signal S_IN, and generates amplified signal V_IN at its output, which is connected to an input of output stage 250. Output stage 250 drives output signal V_OUT at desired voltage and current levels, for communication to transducer 220 via bus 215. The output of output stage 250 is also fed back to an input of linear amplifier 240 to establish a control loop. For example, linear amplifier 240 may receive input signal S_IN at a positive input, and feedback from output signal V_OUT at a negative input. A passive network (e.g., a resistor voltage divider) may couple the output of output stage 250 to the negative input of amplifier 240.

The example of transmitter 210 shown in FIG. 2B includes a single instance of each of linear amplifier 240 and output stage 250. In some implementations, however, transmitter 210 may include multiple instances of linear amplifier 240 and output stage 250 in parallel, for example to individually drive an array of piezoelectric transducer elements in transducer 220.

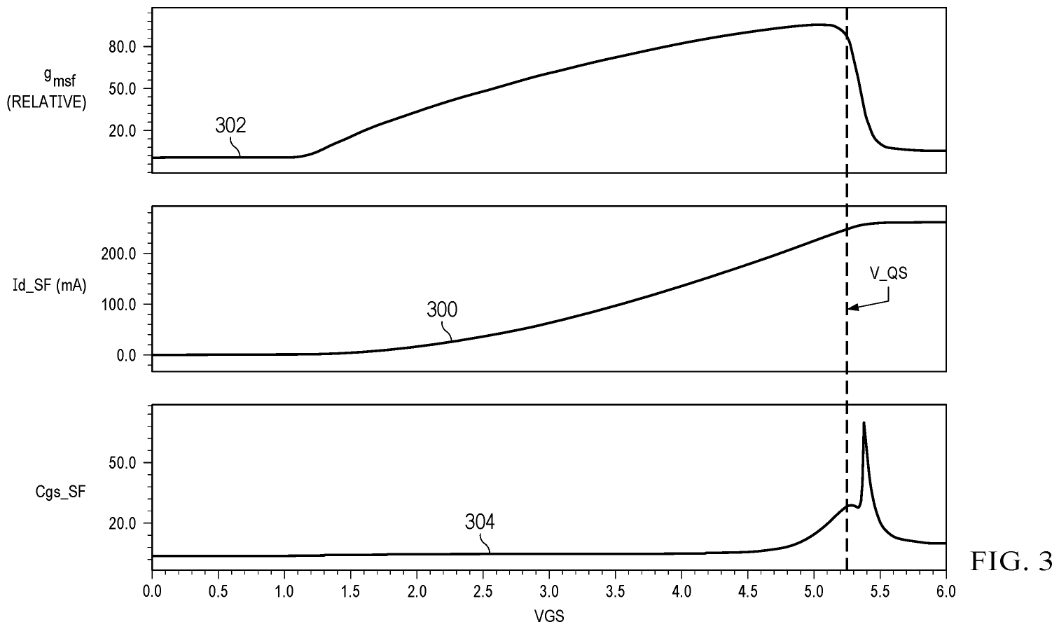

As noted above, the phenomenon of quasi-saturation may be present in MOS transistors when operated at high voltages and high currents. FIG. 3 illustrates an example of this effect as measured in a drain-extended MOS (DEMOS) transistor implemented in a source follower arrangement, for example as NMOS transistor 102N in output stage 100 of FIG. 1A. Plot 300 of FIG. 3 illustrates the drain current (Id_SF) of this transistor with respect to gate-to-source voltage (vgs). As evident from plot 300, drain current Id_SF increases as gate-to-source voltage vgs increases above the transistor threshold voltage (e.g., at about 1.1V in this example) in the saturation region of the transistor. Quasi-saturation occurs in this example upon the gate-to-source voltage vgs reaching and exceeding a relatively high voltage V_QS, e.g., at about 5.25V in FIG. 3. In quasi-saturation, the drain current Id_SF no longer increases as the gate-to-source voltage vgs increases above voltage V_QS.

Plot 302 illustrates the transconductance g_{msf} (in relative terms) of this NMOS source follower transistor with respect to gate-to-source voltage vgs. Plot 302 shows that in the saturation region, the transconductance of the device increases with vgs until quasi-saturation is reached at voltage V_QS. In quasi-saturation, however, the transconductance g_{msf} of the transistor crashes to near zero, because drain current Id_SF no longer increases at gate-to-source voltages beyond voltage V_QS. In addition, plot 304 shows that the gate-to-source capacitance Cgs_SF of the transistor increases dramatically in quasi-saturation, for example by on the order of a factor of two over its capacitance in the saturation region.

It has been observed, in connection with these examples, that the operation of some transmitters at high voltages, high currents, and high frequencies can cause source follower transistors in the transmitter output stage to operate in quasi-saturation region over a significant fraction of its operating cycle. As shown in FIG. 3, the effective source follower transconductance g_{msf} (plot 302) is near zero when the source follower transistors are in quasi-saturation. As shown in FIG. 1B, this near-zero transconductance g_{msf} in quasi-saturation causes the frequency of the non-dominant pole (at a frequency g_{msf}/cₗ) to drastically move to a lower frequency, rather than move to a safer higher frequency as it would with the source follower transistor remaining in saturation. This crashing of the non-dominant pole due to quasi-saturation of the source follower transistors degrades the phase margin of the transmitter, causing instability.

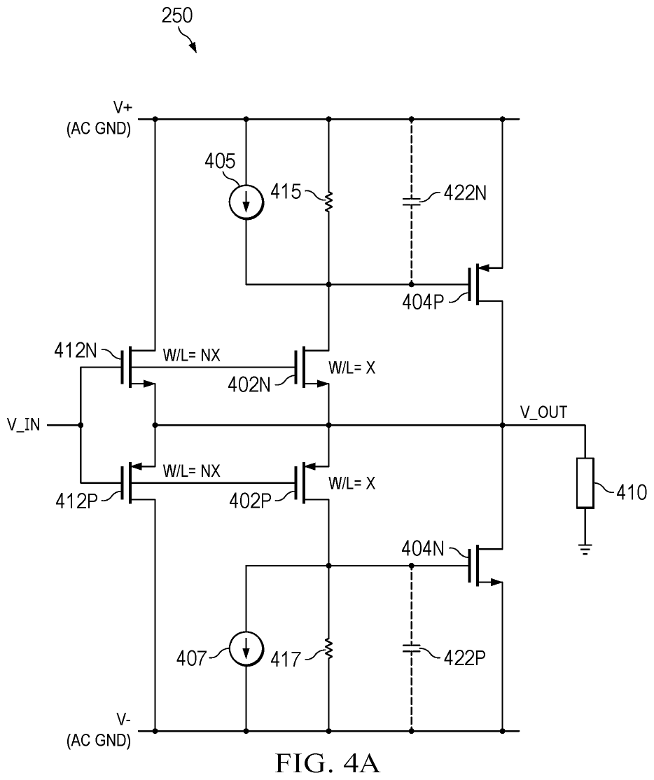

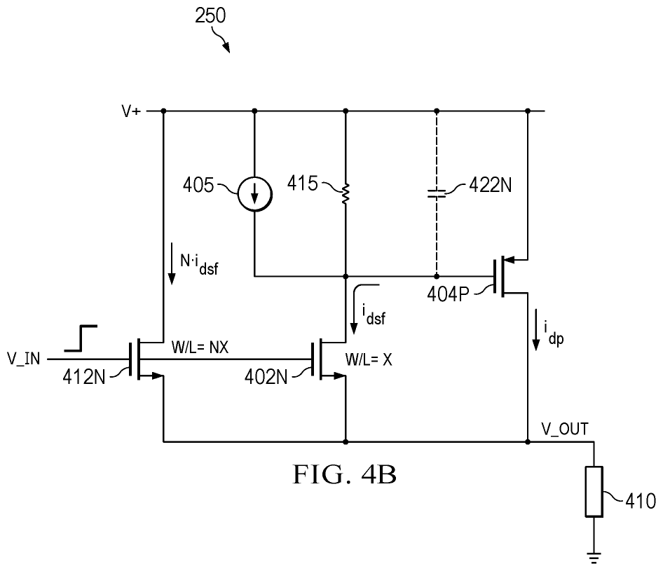

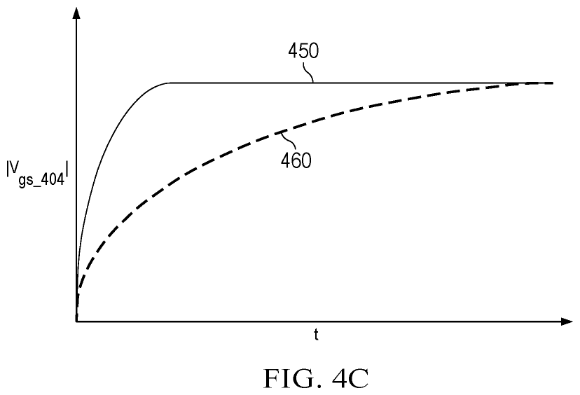

FIG. 4A illustrates the construction of output stage 250 in transmitter 210 according to an example embodiment. Output stage 250 includes NMOS transistors 402N, 404N, and 412N, and PMOS transistors 402P, 404P, 412P. Output stage 250 further includes current sources 405 and 407, and resistors 415 and 417. As shown in FIG. 4A, NMOS transistors 402N and 412N, and PMOS transistors 402P and 412P are implemented in output stage 250 in a source follower arrangement. NMOS transistor 404N and PMOS transistor 404P are implemented at the output of output stage 250. As such, NMOS transistor 404N and PMOS transistor 404P may be constructed to have a drive capability (e.g., channel width to length ratio, or W/L) to drive load 410 at a specified power level. Accordingly, transistors 404N and 404P may be referred to herein as power transistors.

As will be apparent from the following description, output stage 250 is constructed and operable to reduce instability in the output stage caused by quasi-saturation of its source follower transistors. In addition, output stage 250 in this example can reduce the time that its source follower transistors are in quasi-saturation, even when operating at high voltages, high currents, and high frequencies, reducing the potential for instability in the transmitter control loop.

Output stage 250 according to the implementation of FIG. 4A includes NMOS transistor 402N and PMOS transistor 402P arranged as source followers, with their gates coupled in common at input node (or terminal) V_IN and their sources coupled in common to load 410 at output node (or terminal) V_OUT. As such, NMOS transistor 402N and PMOS transistor 402P may be referred to herein as source follower transistors. In the context of an ultrasonic imaging system as in FIG. 2A, load 410 may be largely capacitive, for example with a capacitance of on the order of 300 pF. The drain of NMOS source follower transistor 402N is coupled to the gate of PMOS power transistor 404P, and the drain of PMOS source follower transistor 402P is coupled to the gate of NMOS power transistor 404N. The source of PMOS power transistor 404P is coupled to positive supply terminal V+ and the source of NMOS power transistor 404N is coupled to negative supply terminal V−. For purposes of AC analysis of output stage 250, supply terminals V+, V− can be considered to be at “AC ground”.

Current source 405 biases the drain of NMOS source follower transistor 402N from positive supply terminal V+. Similarly, current source 407 biases the drain of PMOS source follower transistor 402P from negative supply terminal V−. Compensation resistor 415 has a terminal coupled to the drain of NMOS source follower transistor 402N and a terminal coupled to positive supply terminal V+, and similarly compensation resistor 417 has a terminal coupled to the drain of PMOS source follower transistor 402P and a terminal coupled to negative supply terminal V−. According to this implementation, the compensation networks at the drains of source follower transistors 402N, 402P are resistive. In contrast to output stage 100 of FIG. 1A, output stage 250 does not include an additional compensation capacitor either in series or parallel with compensation resistors 415, 417. Capacitance at the drains of source follower transistors 402N, 402P is instead limited to the parasitic gate-to-source capacitances of power transistors 404P and 404N. These parasitic capacitances, shown in shadow as capacitors 422N, 422P, are much smaller (e.g., on the order of 60 pF) than the intentionally added compensation capacitors 114, 116 (e.g., which are on the order of 480 pF) in the output stage of FIG. 1A.

Output stage 250 also includes NMOS transistor 412N and PMOS transistor 412P arranged as source followers, which have their gates coupled in common at input node V_IN and their sources coupled in common at output node V_OUT. The source followers of transistors 412N and 412P are coupled in parallel with source follower transistors 402N and 402P between input node V_IN and output node V_OUT. As such, NMOS transistor 412N and PMOS transistor 412P may be referred to herein as parallel source follower transistors. The drain of NMOS parallel source follower transistor 412N is coupled to positive supply terminal V+, and the drain of PMOS parallel source follower transistor 412P is coupled to negative supply terminal V−. In this implementation, parallel source follower transistors 412N, 412P are constructed to have a larger drive than source follower transistors 402N, 402P, respectively. For example, parallel source follower transistors 412N, 412P may have channel width-to-channel length ratios (W/L) that are a multiple of the W/L ratios of their corresponding source follower transistors 402N, 402P, respectively. In one implementation, the W/L ratios of parallel source follower transistors 412N, 412P may be a factor of eight larger than the W/L ratios of corresponding source follower transistors 402N, 402P. The particular relationship of the W/L ratios may depend on the particular implementation of output stage 250. However, it is contemplated that parallel source follower transistors 412N, 412P are substantially larger than the corresponding source follower transistors 402N, 402P.

As mentioned above, output stage 250 is constructed to provide improved stability in transmitter circuits that operate at high voltages, high currents, and high frequencies, and that are thus vulnerable to quasi-saturation effects in the source follower devices. For example, positive supply terminal V+ may be at a voltage as high as +100 VDC, and negative supply terminal V− may be at a corresponding voltage as negative as −100 VDC. In the example of FIG. 4A, each of the transistors in output stage 250 may be constructed to withstand these high voltages (e.g., to withstand higher voltages than logic transistors in the same integrated circuit). For example, the transistors in output stage may be implemented as drain-extended MOS (DEMOS) transistors.

Referring to FIG. 4B, the operation of the top half of output stage 250 when sourcing load current in response to the signal at input node V_IN will be described by way of example. The lower half of output stage 250 of FIG. 4A responds similarly, but in a converse fashion, when sinking load current in response to the input signal. As such, output stage 250 of FIG. 4A operates in “class AB” mode in response to an AC input signal ranging between positive and negative supply voltages. Alternatively, for example if the input and output signals are single-ended in the sense of varying between a ground level (0V) and a positive voltage, the output stage may be constructed according to the example of FIG. 4B with only single-sided source follower and power transistors.

In operation, NMOS source follower transistor 402N turns on in response to a rising half-cycle at input node V_IN, and conducts current i_{dsf} into load 410 at output node V_OUT. At the initial portion of this rising half-cycle, PMOS power transistor 404P is in an off state, as its gate voltage is near its source voltage (e.g., at or near V+). The current i_{dsf} conducted by NMOS source follower transistor 402N in response to the input signal discharges the gate of PMOS power transistor 404P, turning transistor 404P on at such time as its gate-to-source voltage exceeds its threshold voltage. NMOS source follower transistor 402N sources current i_{dsf} into load 410 during this interval before PMOS power transistor 404P turns on. If this current i_{dsf} is sufficiently high, NMOS source follower transistor 402N can enter quasi-saturation during this interval.

In this example implementation, however, the capacitance at the drain of NMOS source follower transistor 402N in output stage 250 is much smaller than that in output stage 100 of FIG. 1A. This is due to the absence of an additional compensation capacitor (e.g., compensation capacitor 115) in output stage 250 at this node. For example, the capacitance at this node in output stage 250 may be on the order of one-eighth or smaller than that in output stage 100 with the added compensation capacitor (e.g., 60 pF parasitic gate-to-source capacitance vs. a 480 pF compensation capacitor). Curve 450 in FIG. 4C illustrates the gate-to-source voltage of PMOS power transistor 404P in output stage 250 as it turns on. This turn-on of the power transistor in output stage 250 is much faster, in a relative sense, than the turn-on of power transistors in output stage 100, shown by curve 460. This faster turn-on characteristic of output stage 250 is due in substantial part to the reduced capacitance at the drain of NMOS source follower 402N.

By eliminating the added compensation capacitor at the drain of NMOS source follower 402N, the bandwidth of the loop between the output and input of output stage 250 (e.g., the loop between output node V_OUT and the gate of PMOS power transistor 404P, through NMOS source follower transistor 402N) effectively increases by a factor corresponding to the reduction in capacitance at the source follower drain. This faster response in turning on PMOS power transistor 404P reduces the time that NMOS source follower transistor 402N is sourcing the larger portion of the load current, and thus can reduce the time that NMOS source follower transistor 402N is in quasi-saturation.

However, it has been observed in connection with these example embodiments that this increase in bandwidth of the output stage can cause instability in practice, for example due to the inductances in substrate power supply routing and other such factors that arise from implementation of transmitter 210 in an integrated circuit. In some implementations, it is undesirable for this inner loop bandwidth of output stage 250 to exceed ten times the operating signal frequency f_{T} of the transmitter. According to the example of output stage 250 as shown in FIG. 4A and FIG. 4B, the presence of parallel source follower transistors 412N, 412P limits this inner loop bandwidth to provide the desired stability, while still attaining significant reduction in quasi-saturation time of source follower transistors 402N, 402P.

Referring to FIG. 4B, NMOS parallel source follower transistor 412N turns on along with NMOS source follower transistor 402N in response to a rising half-cycle at input node V_IN. When both source follower transistors are turned on, NMOS parallel source follower transistor 412N conducts a much larger share of the load current than does NMOS source follower transistor 402N, in this example by a factor N corresponding to the relative W/L ratios of these devices. From the standpoint of loop stability, NMOS parallel source follower transistor 412N attenuates the effective transconductance of PMOS power transistor 404P in the inner loop of output stage 250, which correspondingly can reduce the bandwidth of this inner loop to the extent necessary for the desired stability.

According to these examples, the provision of one or more parallel source follower transistors in a transmitter output stage enables improved stability, particularly in applications such as ultrasonic imaging systems that require the output signal to be at high voltages, high currents, and high frequencies. The parallel source follower transistors according to these examples enable reduction if not elimination of compensation capacitance at gate nodes of the output power transistors, which reduces the time that the source follower transistors at the gate nodes may be in quasi-saturation. The effect of an increase in the inner loop bandwidth of the output stage resulting from this reduction in gate capacitance is compensated by the participation of the parallel source follower transistors in signal conduction to the transmitter output, enabling loop stability to be maintained.

In addition, the output stage according to these examples can be efficiently implemented in integrated circuits. Because the parallel source follower transistors participate in signal conduction with significant drive capability (e.g., at a multiple of the W/L ratio of the source follower devices), the size of the power output transistors can be reduced accordingly, mitigating any cost in chip area required for the parallel transistors. In addition, chip area can be saved by the elimination of compensation capacitors at the power transistor gate nodes, for example saving on the order of 0.5 mm² in chip area per capacitor (e.g., when implemented as polysilicon-to-n-type well capacitors).

As used herein, the terms “terminal”, “node”, “interconnection” and “pin” are used interchangeably. Unless specifically stated to the contrary, these terms are generally used to mean an interconnection between or a terminus of a device element, a circuit element, an integrated circuit, a device, or other electronics or semiconductor component.

Unless otherwise stated, “about,” “approximately,” or “substantially” preceding a value means+/−10 percent of the stated value.

A device that is “configured to” perform a task or function may be configured (e.g., programmed and/or hardwired) at a time of manufacturing by a manufacturer to perform the function and/or may be configurable (or re-configurable) by a user after manufacturing to perform the function and/or other additional or alternative functions. The configuring may be through firmware and/or software programming of the device, through a construction and/or layout of hardware components and interconnections of the device, or a combination thereof.

A circuit or device that is described herein as including certain components may instead be adapted to be coupled to those components to form the described circuitry or device. For example, a structure described as including one or more semiconductor elements (such as transistors), one or more passive elements (such as resistors, capacitors, and/or inductors), and/or one or more sources (such as voltage and/or current sources) may instead include only the semiconductor elements within a single physical device (e.g., a semiconductor die and/or integrated circuit (IC) package) and may be adapted to be coupled to at least some of the passive elements and/or the sources to form the described structure either at a time of manufacture or after a time of manufacture, for example, by an end-user and/or a third-party. While, in some example embodiments, certain elements are included in an integrated circuit and other elements are external to the integrated circuit, in other example embodiments, additional or fewer features may be incorporated into the integrated circuit. In addition, some or all of the features illustrated as being external to the integrated circuit may be included in the integrated circuit and/or some features illustrated as being internal to the integrated circuit may be incorporated outside of the integrated. As used herein, the term “integrated circuit” means one or more circuits that are: (i) incorporated in/over a semiconductor substrate; (ii) incorporated in a single semiconductor package; (iii) incorporated into the same module; and/or (iv) incorporated in/on the same printed circuit board.

Circuits described herein are reconfigurable to include the replaced components to provide functionality at least partially similar to functionality available prior to the component replacement. Components shown as resistors, unless otherwise stated, are generally representative of any one or more elements coupled in series and/or parallel to provide an amount of impedance represented by the shown resistor. For example, a resistor or capacitor shown and described herein as a single component may instead be multiple resistors or capacitors, respectively, coupled in parallel between the same nodes. For example, a resistor or capacitor shown and described herein as a single component may instead be multiple resistors or capacitors, respectively, coupled in series between the same two nodes as the single resistor or capacitor.

Uses of the phrase “ground” in the foregoing description include a chassis ground, an Earth ground, a floating ground, a virtual ground, a digital ground, a common ground, and/or any other form of ground connection applicable to, or suitable for, the teachings of this description.

Modifications are possible in the described examples, and other examples are possible, within the scope of the claims. While one or more embodiments have been described in this specification, it is of course contemplated that modifications of, and alternatives to, these embodiments, such modifications and alternatives capable of obtaining one or more of the technical effects of these embodiments, will be apparent to those of ordinary skill in the art having reference to this specification and its drawings. It is contemplated that such modifications and alternatives are within the scope of the claims presented herein.

What is claimed is:

1. Transmitter circuitry, comprising:
an amplifier having an output; and
an output stage having an input terminal coupled to the output of the amplifier, having an output terminal, and comprising:
a first transistor having a drain coupled to a first supply terminal, a gate coupled to the input terminal, and a source coupled to the output terminal;
a second transistor having a source coupled to the first supply terminal, a gate coupled to the drain of the first source follower transistor, and a drain coupled to the output terminal; and
a third transistor having a drain coupled to the first supply terminal, a gate coupled to the input terminal, and a source coupled to the output terminal.

2. The transmitter circuitry of claim 1, wherein the third transistor is larger than the first transistor.

3. The transmitter circuitry of claim 1, wherein the third transistor is larger than the first transistor by a factor of about eight.

4. The transmitter circuitry of claim 1, wherein the output stage further comprises:
a first resistor having a first terminal coupled to the first supply terminal, and having a second terminal coupled to the gate of the second transistor and the drain of the first transistor.

5. The transmitter circuitry of claim 1, wherein the output stage further comprises:
a fourth transistor having a drain coupled to a second supply terminal, a gate coupled to the input terminal, and a source coupled to the output terminal;
a fifth transistor having a source coupled to the second supply terminal, a gate coupled to the drain of the fourth transistor, and a drain coupled to the output terminal; and
a sixth transistor having a drain coupled to the second supply terminal, a gate coupled to the input terminal, and a source coupled to the output terminal.

6. The transmitter circuitry of claim 5, wherein the sixth transistor is larger than the fourth transistor.

7. The transmitter circuitry of claim 5, wherein the output stage further comprises:
a second resistor having a first terminal coupled to the second supply terminal, and having a second terminal coupled to the gate of the fifth transistor and the drain of the fourth transistor.

8. The transmitter circuitry of claim 5, wherein the first transistor and the third transistor are metal-oxide-semiconductor (MOS) transistors of a first channel conductivity type;
and wherein the fourth transistor and the sixth transistor are MOS transistors of a second channel conductivity type.

9. The transmitter circuitry of claim 8, wherein the second transistor is a MOS transistor of the second conductivity type, and the fifth transistor is a MOS transistor of the first conductivity type.

10. The transmitter circuitry of claim 8, wherein the first, second, third, fourth, fifth, and sixth transistors are constructed as drain-extended MOS transistors.

11. An ultrasonic transceiver system, comprising:
a transducer;
ultrasonic receiver circuitry, coupled to the transducer;
ultrasonic transmitter circuitry, having an output terminal coupled to the transducer;
a transmit/receive switch, coupled to the ultrasonic transmitter circuitry and to the ultrasonic receiver circuitry, and configured to isolate the ultrasonic receiver circuitry from the ultrasonic transmitter circuitry while the ultrasonic transmitter circuitry is transmitting;
wherein the ultrasonic transmitter circuitry comprises:
an amplifier having an output; and
an output stage having an input terminal coupled to the output of the amplifier, and comprising:
a first transistor having a drain coupled to a first supply terminal, a gate coupled to the input terminal, and a source coupled to the output terminal;
a second transistor having a source coupled to the first supply terminal, a gate coupled to the drain of the first transistor, and a drain coupled to the output terminal; and
a third transistor having a drain coupled to the first supply terminal, a gate coupled to the input terminal, and a source coupled to the output terminal;
and wherein the third transistor is larger than the first transistor.

12. The transceiver system of claim 11, wherein the output stage further comprises:
a fourth transistor having a drain coupled to a second supply terminal, a gate coupled to the input terminal, and a source coupled to the output terminal;
a fifth transistor having a source coupled to the second supply terminal, a gate coupled to the drain of the fourth transistor, and a drain coupled to the output terminal; and
a sixth transistor having a drain coupled to the second supply terminal, a gate coupled to the input terminal, and a source coupled to the output terminal;
and wherein the sixth transistor is larger than the fourth transistor.

13. The transceiver system of claim 12, wherein the output stage further comprises:
a first resistor having a first terminal coupled to the first supply terminal, and having a second terminal coupled to the gate of the second transistor and the drain of the first transistor; and
a second resistor having a first terminal coupled to the second supply terminal, and having a second terminal coupled to the gate of the fifth transistor and the drain of the fourth transistor.

14. The transceiver system of claim 12, wherein the first transistor, the third transistor, and the fifth transistor are metal-oxide-semiconductor (MOS) transistors of a first channel conductivity type;
and wherein the fourth transistor, the sixth transistor, and the second transistor are MOS transistors of a second channel conductivity type.

15. The transceiver system of claim 12, wherein the first, second, third, fourth, fifth, and sixth transistors are constructed as drain-extended MOS transistors.

16. Output stage circuitry, comprising:
a first transistor having a drain coupled to a first supply terminal, a gate coupled to an input terminal, and a source coupled to the output terminal;
a second transistor having a source coupled to the first supply terminal, a gate coupled to the drain of the first source follower transistor, and a drain coupled to the output terminal; and
a third transistor having a drain coupled to the first supply terminal, a gate coupled to the input terminal, and a source coupled to the output terminal;
wherein the third transistor is larger than the first transistor.

17. The output stage circuitry of claim 16, further comprising:
a fourth transistor having a drain coupled to a second supply terminal, a gate coupled to the input terminal, and a source coupled to the output terminal;
a fifth transistor having a source coupled to the second supply terminal, a gate coupled to the drain of the second source follower transistor, and a drain coupled to the output terminal; and
a sixth follower transistor having a drain coupled to the second supply terminal, a gate coupled to the input terminal, and a source coupled to the output terminal;
wherein the sixth transistor is larger than the fourth transistor.

18. The output stage circuitry of claim 17, further comprising:
a first resistor having a first terminal coupled to the first supply terminal, and having a second terminal coupled to the gate of the second transistor and the drain of the first transistor; and
a second resistor having a first terminal coupled to the second supply terminal, and having a second terminal coupled to the gate of the fifth transistor and the drain of the fourth transistor.

19. The output stage circuitry of claim 18, wherein the first transistor, the third transistor, and the fifth transistor are metal-oxide-semiconductor (MOS) transistors of a first channel conductivity type;
and wherein the fourth transistor, the sixth transistor, and the second transistor are MOS transistors of a second channel conductivity type.

20. The output stage circuitry of claim 18, wherein the first, second, third, fourth, fifth, and sixth transistors are constructed as drain-extended MOS transistors.
